(19) United States
(12) Patent Application Publication
(10) Pub. No.: US 2011/0287531 A1
(43) Pub. Date: Nov. 24, 2011

(54) Microalgae Growth Pond Design

(76) Inventor: David A. Hazlebeck, El Cajon, CA (US)

(21) Appl. No.: 12/784,338

(22) Filed: May 20, 2010

(51) Int. Cl. C12M 1/02

(52) U.S. Cl. 435/289.1

(57) ABSTRACT

A raceway pond for circulating microalgae in a fluid medium includes a plurality of interconnected channels. Each channel is straight and has a structured gradient, due to tilt or terracing, that moves the fluid medium along the raceway. In operation, the concentration of microalgae in the fluid medium is maintained substantially constant, and the depth of the fluid medium in the raceway is maintained below a pre-determined level.

### FIELD OF THE INVENTION

The present invention pertains generally to plug-flow reactors (PFRs) having a circulating raceway pond for growing microalgae in a fluid medium. More particularly, the present invention pertains to PFRs that provide conditions for producing microalgae having an oil content as high as 60%. The present invention is particularly, but not exclusively, useful as a PFR that relies on gravity for moving microalgae in a fluid medium along the length of its raceway.

### BACKGROUND OF THE INVENTION

The growth rate of microalgae in a liquid environment is dependent on several disparate factors. For one, it is known that the fluid medium in which the microalgae grows (i.e. liquid environment) must be circulated to provide for mixing and exposure of the microalgae to light for photosynthesis. For another, each algae species has an optimal concentration for consumption of all, or nearly all, of the available resources in the fluid medium. The import here is that with a high consumption of available resources by the microalgae, the time available for growth of weed algae, bacteria or predators that would otherwise diminish algae production, is limited. Yet another factor concerns the depth of a circulating microalgae pond. Indeed, pond depth has been determined to be a very important factor affecting microalgae growth.

Heretofore, conventional thinking has been that an increase in the net production of microalgae in a circulating pond could be achieved only by an increase in the depth of the fluid medium in the pond. It has been determined, however, this is not the case. Contrary to earlier conclusions, shallow circulating pond depths of around 7.5 to 10 cm have proven more efficient and more productive than deeper ponds. A problem with shallow ponds, however, is that typical means used for circulating the fluid medium are generally ineffective when pond depths are in the 7.5-10 cm range. For example, paddle wheels are typically not effective for this purpose with pond depths less than 20 cm.

Another factor for consideration, when designing a system that will be used to grow microalgae for commercial purposes, is the volume of microalgae that can be produced. On this point, it is clear that the amount of biomass that can be produced is directly proportional to the volume of fluid medium that can be used. There must, of course, be compliance with the pond depth and concentration considerations mentioned above. Nevertheless, although a shallow depth for the fluid medium is crucial, the width of fluid channels that are constructed for the circulating pond is not so limited.

Yet another factor for consideration, when using a conventional raceway circulating pond having a paddle wheel to grow microalgae, is that the pond size is limited to be under several acres to maintain relative evenness. On this point, the larger the area, the more the unevenness in depth across the culture area, which can be a drawback in terms of productivity.

In light of the above, it is an object of the present invention is to provide a circulating pond that is dimensioned to provide conditions for optimal growth of microalgae. Another object of the present invention is to provide a circulating pond with raceways that avoid dead zones, and consequently uneven fluid flow. Still another object of the present invention is to provide a circulating pond for promoting microalgae growth that relies on gravity as the primary force for moving a fluid medium through the pond. Yet another object of the present invention is to provide a circulating pond that is relatively simple to manufacture, is easy to use, and is comparatively cost effective.

### SUMMARY OF THE INVENTION

A raceway pond that is used to circulate a fluid medium for the purpose of growing algae includes a pair of substantially straight, elongated channels. The channels are generally juxtaposed, side-by-side to each other, with both of their respective ends in fluid communication with each other. Several structural aspects of the channels are of particular importance. For one, the fluid medium in the channels has a substantially constant and relatively shallow depth (e.g. 7.5 cm). For another, the channels have a structured downstream gradient that allows fluid to flow continuously from the upstream end of one channel to the downstream end of the other channel under the influence of gravity.

In detail, each elongated channel of the raceway pond has a first (upstream) end and a second (downstream) end, with a substantially flat floor and opposed sidewall portions extending between the ends. For disclosure purposes, one channel is referred to hereinafter as the first channel, and the other channel is referred to as the second channel. A transfer section connects the second (downstream) end of the first channel in fluid communication with the first (upstream) end of the second channel. Importantly, this transfer section provides for a gravity flow of the fluid medium from the first channel to the second channel. At the second (downstream) end of the second channel, a lifting device is provided to lift water from the second (downstream) end of the second channel, back into the first (upstream) end of the first channel. For purposes of the present invention, the lifting device can be of any type well known in the pertinent art and is, preferably, selected from a group consisting of an Archimedes pump, a conveyor, a bucket lift, a paddle wheel, a sealed paddle wheel or an electro-mechanical pump.

As indicated above, a structured downstream gradient is provided for each channel that will cause the fluid medium to flow through the raceway pond under the influence of gravity. In one configuration for this structured downstream gradient, the floor of the channel is provided with an incline. For example, a 1.3 foot height difference between the ends of a 2500 foot long channel would provide an adequate incline for the present invention. Alternatively, the structured gradient can be accomplished by constructing steps along the length of the floor of a channel. If steps are used, each step could be formed with a height “h” of approximately 3 cm, with a distance “s” between steps of approximately 100 m. Further, for either floor configuration, a plurality of vortex generators can be mounted on the floor in the channel to create turbulence in the fluid medium that will assist algae growth.

In addition to the structural aspects of the channels mentioned above, the sidewall portions of the first channel can be tapered with an increasing downstream width established by a taper angle “α” that is equal to approximately 0.002 radians. With this taper, the first channel will establish a logarithmic growth stage for microalgae in the raceway pond. The sidewalls of the second channel can then be oriented substantially parallel to each other to provide for an oil accumulation stage for the microalgae.

An important aspect of the present invention is its scale. In particular, this aspect concerns the physical dimensions of the first and second channels. For example, each channel can have a length of approximately 2,500 m, and a width that can be greater than about 100 m. Further, regardless of other dimensions for the raceway pond, it is important that the depth of fluid medium in the channels be maintained below a level of about 15 cm. And, preferably, the depth of fluid medium will be around 7.5 cm.

At least one injector can be provided with the raceway pond to add fluid medium to the pond at a selected point(s) along the length of the raceway. The purpose for adding the fluid medium is two-fold. First, the addition of fluid medium is done to maintain the depth of the fluid medium substantially constant in the channels (e.g. 7.5 cm). Second, the controlled addition of fluid medium, together with the tapered construction of the first channel, provide for the maintenance of a pre-determined concentration of microalgae in the fluid medium (e.g. approximately 1.5 grams per liter). These considerations, along with the dimensions and structural aspects given above for the channels, are intended to ensure an operational net oil productivity from algae grown in the raceway pond that is in a range of 15-50 g/m²/day.

### BRIEF DESCRIPTION OF THE DRAWINGS

The novel features of this invention, as well as the invention itself, both as to its structure and its operation, will be best understood from the accompanying drawings, taken in conjunction with the accompanying description, in which similar reference characters refer to similar parts, and in which:

FIG. 1 is a top view of a circulating pond in accordance with the present invention;

FIG. 2A is a side cross-section view of a structured gradient for the raceway of the present invention as seen along the line 2-2 in FIG. 1;

FIG. 2B is a side cross-section view of an alternate embodiment for the structured gradient of the raceway, as would be seen along the line 2-2 in FIG. 1; and

FIG. 3 is a top view of an alternate embodiment for a circulating pond for the present invention.

### DESCRIPTION OF THE PREFERRED EMBODIMENTS

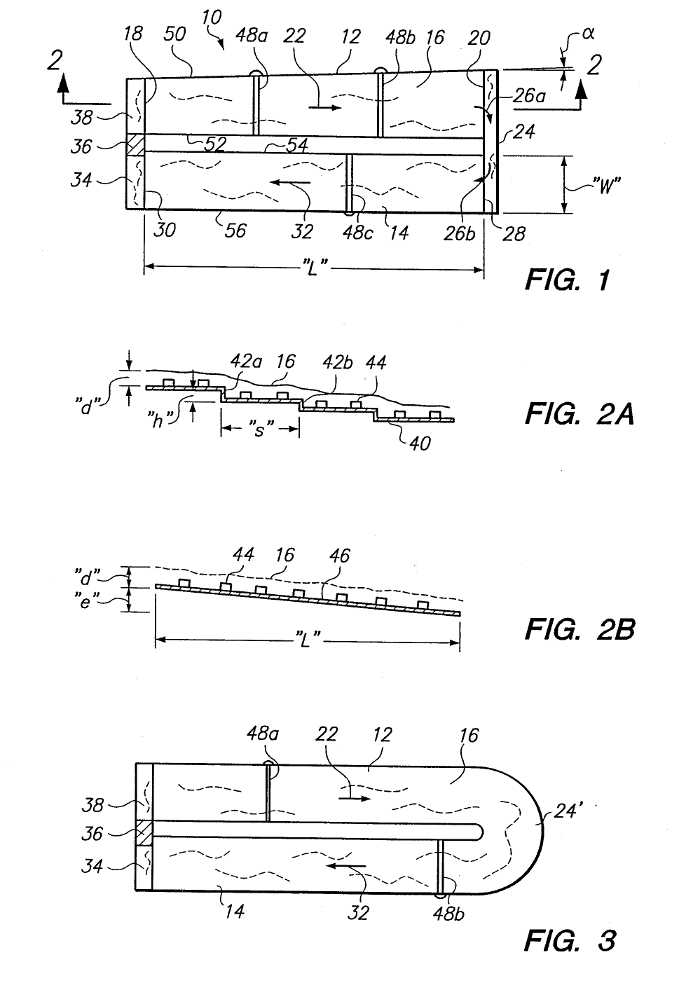

Referring initially to FIG. 1 a raceway pond in accordance with the present invention is shown and is generally designated 10. Specifically, in FIG. 1 it can be seen the pond 10 includes a first channel 12 and a second channel 14 that are shown juxtaposed in a side-by-side relationship with one another. Further, it is shown that the channels 12 and 14 are in fluid communication with each other and that a fluid medium 16 flows continuously from one to the other. As will be appreciated by the skilled artisan, the arrangement of the channels 12 and 14 shown in FIG. 1 is only exemplary. Depending on topography of the terrain where the pond 10 will be used, and the ability to satisfy other requirements of the present invention, the channels 12 and 14 can have any of various arrangements.

In greater detail, FIG. 1 shows that the fluid medium 16 flows in the first channel 12 from an upstream end 18 to a downstream end 20, as indicated by the arrow 22. After flowing through the first channel 12, the fluid medium 16 transitions through a transfer section 24 from the first channel 12 to the second channel 14, as indicated by the arrows 26a and 26b. In the second channel 14, the fluid medium 16 flows from an upstream end 28 to a downstream end 30, as indicated by the arrow 32. At the downstream end 30 of the second channel 14, the fluid medium 16 enters a collection trough 34. A lifting device 36 is then used to lift the fluid medium 16 from the collection trough 34 (channel 14) and into a distribution trough 38 (channel 12). As envisioned for the present invention, the algae culture will pass through the circulation pump (e.g. lifting device 36) every 2-4 hours. As the cell size is generally small (1-20 μm dia.) and may have a thick cell wall, the shear stress generated by the pump (lifting device 36) has little or no effect on growth. However, to insect larva, the shear stress is significant as larva is generally large in size (10 mm) and has no cell wall. Therefore, such design also helps prevent contamination of the algae culture by insects. Moreover, for an open body of water, such design is environmentally friendly due to this insect control mechanism. Thus, fluid medium 16 is transferred from the downstream end 30 of the second channel 14 to the upstream end 18 of the first channel 12 for a re-circulation of the fluid medium 16 through the raceway pond 10. Preferably, the lifting device 36 is of a type well known in the pertinent art, such as a conveyor, a bucket lift, a paddle wheel, a sealed paddle wheel or an electro-mechanical pump.

As implied above, except for the lifting device 36 between the collection trough 34 (channel 14) and the distribution trough 38 (channel 12), the fluid medium 16 flows through the entire pond 10 under the influence of gravity. For purposes of the present invention, this gravity flow is accomplished using a structured gradient. A preferred embodiment of a structured gradient for use with the pond 10 is shown in FIG. 2A. There it will be seen that the respective floors 40 of channel 12 and 14 are formed with a plurality of steps 42 (the steps 42a and 42b are exemplary). In detail, the steps 42 are defined by a height “h” of approximately 3 centimeters, with a distance “s” between the steps 42 being preferably on the order of approximately 100 meters. FIG. 2A also shows that a plurality of vortex generators 44 can be positioned along the respective floors 40 of the channels 12 and 14 for the purpose of providing turbulent flow for the fluid medium 16.

In an alternate embodiment of a structured gradient as shown in FIG. 2B, a floor 46 is provided with an incline. For example, the slope of this incline will be “e/L”, as indicated in FIG. 2B. And, “e” will preferably equal about one meter, and “L” will equal about 2,500 meters. Importantly, although the dimensions of the incline can change, a desired volumetric flow rate is provided by the incline in all instances. Again, vortex generators 44 can be employed. Impliedly, the dimensions given here are approximate, and are given to provide a notion of scale for the invention. Accordingly, actual dimensions can be selected to suit the individual needs of the raceway pond 10.

An important aspect of the raceway pond 10 for the present invention will be appreciated with reference to FIG. 2A, and again to FIG. 1. This aspect is that the depth “d” of the fluid medium 16 in the channels 12 and 14 needs to be rather shallow (i.e. less than about 15 cm, and preferably around 7.5 cm). To maintain this depth “d”, however, it may be necessary to replenish the fluid medium 16 along the lengths “L” of the channels 12 and 14. This may be for any of several reasons (e.g. evaporation losses). Regardless of the reason, however, replenishment can be done by appropriately positioning injectors 48 along the channels 12 and 14 (injectors 48a, 48b and 48c are only exemplary).

For an operation of the present invention, microalgae (not shown) are to be grown in the pond 10. For this purpose, it is necessary the pond 10 have a logarithmic growth stage (i.e. channel 12), as well as an oil accumulation stage (i.e. channel 14). The logarithmic growth stage, however, needs to be constructed with a configuration that will accommodate growth of the microalgae. Accordingly, the side 50 of channel 12 can be slightly angled relative to the side 52 of the channel 12, to thereby provide an increasing taper for the channel 12 from its upstream end 18 to its downstream end 20. It happens that, due to the relatively extreme length of the channel 12, the magnitude of the taper angle “α” that is needed to do this will be on the order of only approximately 0.002 radians. With this in mind, the purpose of adding fluid medium 16 from injectors 48 into the logarithmic growth stage (i.e. channel 12) becomes two-fold. In addition to maintaining a substantially constant depth “d” of fluid medium 16 in channel 12, the addition of fluid medium 16 can be controlled to maintain a pre-determined concentration of the microalgae in the fluid medium 16. Preferably, this pre-determined concentration is approximately 1.5 grams per liter.

Unlike the logarithmic growth stage provided by channel 12, the oil accumulation stage provided by channel 14 is not concerned with microalgae growth, but rather with allowing the microalgae to mature. Accordingly, although the depth “d” needs to be maintained as discussed above, the main concern for channel 14 is to keep the fluid medium 16 moving. This can be done with the respective sides 54 and 56 of the channel 14 being constructed substantially parallel to each other.

For a modification of the raceway pond 10 of the present invention, instead of a configuration for transfer section 24 as shown in FIG. 1, a transfer section 24′ as shown in FIG. 3 can be provided. Specifically, the transfer section 24′ shown in FIG. 3 provides for a continuous turn from channel 12 to channel 14. Regardless of configurations, however, the depth “d” of fluid medium 16 in the raceway pond 10, the pre-determined concentration of microalgae in the fluid medium 16, and the volumetric fluid flow of the fluid medium 16 around the raceway pond 10 are each calculated to provide for an operational oil productivity from algae growth that is in a range of approximately 15-50 g/m²/day.

While the particular Microalgae Growth Pond Design as herein shown and disclosed in detail is fully capable of obtaining the objects and providing the advantages herein before stated, it is to be understood that it is merely illustrative of the presently preferred embodiments of the invention and that no limitations are intended to the details of construction or design herein shown other than as described in the appended claims.

What is claimed is:

1. A raceway pond for circulating a fluid medium which comprises:
a substantially straight, elongated channel having a first end with a wall and a second end with a wall, with opposed sidewall portions extending therebetween, wherein the first channel includes a substantially flat floor to establish a substantially rectangular cross section for fluid flow, and the floor has a structured downstream gradient from the first end to the second end of the channel; and
at least one injector means for adding fluid medium into the pond at a selected point, to maintain a substantially constant depth “d” for fluid medium in the channel during circulation, wherein “d” is less than an established maximum depth.

2. A pond as recited in claim 1 wherein the channel is a first channel and the pond further comprises:
a second channel, wherein the second channel is substantially straight, is elongated, and has a first end with a wall and a second end with a wall, with opposed sidewall portions extending therebetween, and wherein the second channel includes a substantially flat floor having a structured downstream gradient from its first end to its second end;
a transfer section connecting the second end of the first channel in fluid communication with the first end of the second channel; and
a lifting device positioned to lift fluid medium from the second end of the second channel to the first end of the first channel for circulating the fluid medium through the pond.

3. A pond as recited in claim 1 wherein the channel has a width “w” and the structured gradient is provided by a plurality of steps along the floor of the channel, wherein each step is individually formed with a height “h” and is separated from an adjacent step by a distance “s” along the length “L” of the channel.

4. A pond as recited in claim 3 wherein “w” is greater than 100 m, “h” is approximately 3 cm, “s” is approximately 100 m, “L” is approximately 2500 m, and “d” is approximately 7.5 cm.

5. A pond as recited in claim 1 further comprising a plurality of vortex generators mounted on the floor of the channel to create turbulence in the fluid medium.

6. A pond as recited in claim 1 wherein the lifting means is selected from a group consisting of a conveyor, a bucket lift, a paddle wheel, a sealed paddle wheel and an electro-mechanical pump.

7. A pond as recited in claim 2 wherein the fluid medium includes microalgae and the side wall portions of the first channel form a taper with an increasing width “w” for the first channel in a direction from the first end toward the second end thereof to establish a logarithmic growth stage for the microalgae.

8. A pond as recited in claim 7 wherein the side wall portions of the second channel are substantially parallel to each other to establish an oil accumulation stage for the microalgae.

9. A raceway pond for circulating a fluid medium which comprises:
a first channel, wherein the first channel is substantially straight, is elongated, and has a first end with a wall and a second end with a wall, with opposed sidewall portions extending therebetween, and wherein the first channel includes a floor having a structured downstream gradient from the first end to the second end;
a second channel, wherein the second channel is substantially straight, is elongated, and has a first end with a wall and a second end with a wall, with opposed sidewall portions extending therebetween, and wherein the second channel includes a floor having a structured downstream gradient from its first end to its second end;
a transfer section connecting the second end of the first channel in fluid communication with the first end of the second channel; and
a lifting device positioned to lift fluid medium from the second end of the second channel to the first end of the first channel for circulating the fluid medium through the pond.

10. A pond as recited in claim 9 wherein the lifting means is selected from a group consisting of a conveyor, a bucket lift, a paddle Wheel, a sealed paddle wheel and an electro-mechanical pump.

11. A pond as recited in claim 9 wherein the fluid medium includes microalgae and the side wall portions of the first channel form a taper, defined by an angle “α”, with the taper having an increasing width “w” for the first channel in a direction from the first end toward the second end thereof to establish a logarithmic growth stage for the microalgae.

12. A pond as recited in claim 11 wherein the side wall portions of the second channel are substantially parallel to each other to establish an oil accumulation stage for the microalgae.

13. A pond as recited in claim 11 further comprising a plurality of vortex generators mounted on the respective floors of the first and second channels for creating turbulence in the fluid medium to provide vertical lifting for the microalgae.

14. A pond as recited in claim 11 wherein the structured gradient is provided by a plurality of steps along the floor of the channel, wherein each step is individually formed with a height “h” and is separated from an adjacent step by a distance “s” along the length “L” of the channel.

15. A pond as recited in claim 14 wherein “α” is an angle approximately equal to 0.002 radians, “w” is greater than 100 m, “h” is approximately 3 cm, “s” is approximately 100 m, “L” is approximately 2500 m, and “d” is approximately 7.5 cm.

16. A system for promoting a net oil productivity from microalgae in a fluid medium which comprises:
a plurality of elongated channels connected end-to-end to form a raceway with each channel having an upstream end and a downstream end, wherein each channel has a rectangular cross section, and has a structured downstream gradient for causing a flow of the fluid medium therethrough, and wherein each channel has a downstream end connected to an upstream end of an adjacent channel; and
an injector means for adding fluid medium at selected points along the raceway to promote a microalgae growth rate in the fluid medium by maintaining a concentration of microalgae in the fluid medium below a pre-determined concentration level, and by keeping a depth “d” for fluid medium in the channel during circulation below an established maximum depth.

17. A system as recited in claim 16 wherein the pre-determined concentration level is less than approximately 1.5 grams per liter, wherein “d” is less than approximately 15 cm, and wherein the net productivity is in a range of 15-50 g/m²/day.

18. A system as recited in claim 16 wherein the plurality of channels comprises:
a first channel formed with a taper having an increasing width “w” in a direction from the first end toward the second end thereof to establish a logarithmic growth stage for the microalgae; and
a second channel having a substantially constant width “w” to establish an oil accumulation stage for the microalgae.

19. A system as recited in claim 16 wherein the structured gradient is provided by a plurality of steps along the floor of the channel, and wherein each step is individually formed with a height “h” and is separated from an adjacent step by a distance “s” along the length “L” of the channel.

20. A system as recited in claim 19 wherein “w” is greater than 100 m, “h” is approximately 3 cm, “s” is approximately 100 m, “L” is approximately 2500 m, and “d” is approximately 7.5 cm.
